(19) United States
(12) Patent Application Publication
(10) Pub. No.: US 2024/0021279 A1
(43) Pub. Date: Jan. 18, 2024

(54) SYSTEM FOR PREDICTIVE MEDICAL TREATMENT RECOMMENDATION AND DIGITAL PLATFORM INTEGRATION THEREOF

(71) Applicant: OSMIND INC., San Francisco, CA (US)

(72) Inventors: Matthew Jacob Worley, Austin, TX (US); Jimmy J. Qian, Redwood City, CA (US)

(73) Assignee: OSMIND INC., San Francisco, CA (US)

(21) Appl. No.: 18/221,864

(22) Filed: Jul. 13, 2023

Related U.S. Application Data
(60) Provisional application No. 63/388,968, filed on Jul. 13, 2022.

(51) Int. Cl. G16H 10/60; G16H 50/20; G16H 20/17

(52) U.S. Cl. CPC G16H 10/60; G16H 50/20; G16H 20/17

(57) ABSTRACT

A system for electronic health record (EHR) management and dynamic treatment adherence prediction comprising a set of computer-executable instructions causing the server to generate, via a first client device, a patient-facing interface configured to receive patient information; transmit, from the first client device to the server, the patient information; populate, via the at least one server processor, an EHR of the patient with the patient information; generate, via the at least one server processor, via a treatment adherence algorithm based at least on a portion of the patient information, an adherence probability of the patient, wherein the adherence probability is the likelihood that the patient will complete a treatment; transmit, from the server to a second client device, the EHR and the adherence probability; generate, via the second client device, a provider-facing interface comprising the EHR of the patient, the EHR comprising the adherence probability of the patient.

### CROSS-REFERENCE TO RELATED APPLICATIONS

The present application claims the benefit of U.S. Patent Application No. 63/388,968 for SYSTEM FOR PREDICTIVE MEDICAL TREATMENT RECOMMENDATION AND DIGITAL PLATFORM INTEGRATION THEREOF, filed Jul. 13, 2022, the entire contents of which are incorporated herein by reference in their entirety.

### FIELD OF THE INVENTION

The present disclosure relates to systems and methods for evaluating treatment efficacy. Specifically, the present disclosure relates to systems and methods for integrating predictive models for determining the likelihood of IV ketamine treatment adherence with electronic health record platforms.

### INTRODUCTION

Today, there exist a myriad of digital health suites, electronic health record databases, and other medical platforms and networks. As a result, clinicians and other medical professionals may be required to utilize a multitude of the aforementioned platforms in order to utilize their preferred tools, which may be proprietary to a particular platform or entirely absent from others. Therefore, clinicians, especially those treating mental health conditions such as clinical depression, may be burdened by switching between these platforms. Further, the records as stored on one platform may be incongruent and/or restricted for use with other platforms. Thus, a clinician's ability to most effectively treat a patient in view of these computerized tools is frustrated by the failure to integrate useful tools within such platforms.

Currently, clinical depression is one of the most common and costly health problems in the world. However, the most popular evidence-based treatments for depression do not produce lasting benefits in roughly 30% of the patients who receive them.

Recently, intravenous (IV) ketamine has emerged as a viable option for treatment of depression, especially in cases when other treatments have failed. Clinical studies have demonstrated that IV ketamine typically produces rapid and large reductions in depression when patients receive a full initial course of treatment, i.e., 4-8 infusions within 28 days (the “induction treatment”). Nevertheless, nearly half of patients who start IV ketamine do not adhere to the prescribed visit routine for the induction treatment. This is problematic for both clinicians and patients. Data shows that patients who are adherent to the prescribed regimen have better outcomes than those who stop treatment prematurely or do not receive induction treatment. Clinicians lack data-driven tools to know when to offer IV ketamine treatment versus alternative treatments. Often patients invest significant financial resources and time into IV ketamine treatment that ultimately may not work for them. Such an issue is exacerbated because ketamine for depression is typically not covered by health insurance.

In the current state of IV ketamine clinical practice, a clinician typically conducts an initial baseline evaluation or consultation. After this consultation, the clinician and patient must make an informed decision on whether to begin a course of IV ketamine treatment. To make this decision, clinicians may rely on such factors as whether the patient has uncontrolled hypertension or other neurological or cardiac conditions that could be aggravated by elevated blood pressure, which is a common side-effect of ketamine. Liver enzymes that are three times more prevalent than that of normal levels are a relative contraindication. In terms of psychiatric conditions, psychosis is typically the only condition that is contraindicated. Additionally, patients cannot be pregnant or breastfeeding. Cost is often a prohibitive factor and ketamine IV treatment (“KIT”) may be relatively contraindicated if another treatment is available and the patient can get reimbursed for care. An important factor that would incline a clinician to choose KIT over transcranial magnetic stimulation (“TMS”) or esketamine would be the presence of suicidal ideation as KIT is the most rapid acting of these treatments potentially bringing relief within weeks.

Traditionally, some clinicians may be aware of individual factors linked to IV ketamine outcomes and may make high-level assumptions of treatment success. However, human clinicians lack the decision-making capacity to weigh multiple factors simultaneously and assign the appropriate weight to each individual factor in an algorithmic manner.

Further, traditional electronic health record platforms may collect an impressive quantity and quality of data yet may fail to utilize such data in a meaningful way to assist a clinician. For example, mere storage and retrieval of information may do little to increase the clinician's ability to treat their patient.

It would be desirable to provide a system configured to predict a patient's treatment adherence and/or compliance. It would further be desirable to provide a system to evaluate likelihood of treatment completion in view of patient characteristics extracted before prescription of said treatment. It would yet be further desirable to integrate the predicted probability system with an electronic health record platform, as to streamline the prediction process, beginning at patient intake and providing a set of useful tools accessible to the clinician at multiple potential stages of interaction with the patient.

### SUMMARY

In an aspect of the present disclosure, a system for electronic health record (EHR) management and dynamic treatment adherence prediction, may comprise a server comprising at least one server processor, at least one server database, at least one server memory comprising a set of computer-executable server instructions which, when executed by the at least one server processor, cause the server to: generate, via a first client device, a patient-facing interface configured to receive patient information from a patient; receive, via the first client device, the patient information; transmit, from the first client device to the server, the patient information; populate, via the at least one server processor, an EHR of the patient with the patient information; generate, via the at least one server processor, via a treatment adherence algorithm based at least on a portion of the patient information, an adherence probability of the patient, wherein the adherence probability of the patient is the likelihood that the patient will complete a treatment; transmit, from the server to a second client device, the EHR of the patient and the adherence probability of the patient; generate, via the second client device, a provider-facing interface comprising the EHR of the patient, the EHR of the patient comprising the adherence probability of the patient. In a further embodiment, the set of computer-executable server instructions which, when executed by the at least one server processor, further cause the server to: generate, via the at least one server processor, via a secondary treatment adherence algorithm based at least on a portion of the patient information, a secondary adherence probability of the patient, wherein the secondary adherence probability of the patient is the likelihood that the patient will complete a secondary treatment; transmit, from the server to the second client device, the secondary adherence probability of the patient; generate, via the second client device, the provider-facing interface comprising the EHR of the patient, the EHR of the patient comprising the adherence probability of the patient and the secondary adherence probability of the patient. In yet a further embodiment, the treatment and the secondary treatment are configured to treat a same ailment.

In an embodiment, the treatment comprises intravenous (IV) ketamine infusions. In one embodiment, the completeness of the treatment is defined as the patient completing at least four IV ketamine infusions within twenty-eight days from an intake evaluation.

In an alternative embodiment, the treatment adherence algorithm is selected from a group of classifier types comprising Bayesian linear models, hierarchical models, naive Bayes classifiers, and kernel-based methods.

In an embodiment, the patient information is appended by one or more external sources. In a further embodiment, the one or more external sources comprise a digital biomarker-capturing device configured to capture one or more digital biomarkers from the patient. In yet a further embodiment, the digital biomarker-capturing device is integrated within the first client device.

In another embodiment, the set of computer-executable server instructions which, when executed by the at least one server processor, further cause the server to: detect, via the server processor, one or more changes to the patient information; regenerate, triggered by the one or more changes to the patient information, via the at least one server processor, via the treatment adherence algorithm based at least on the portion of the patient information, the adherence probability of the patient. Further, the set of computer-executable server instructions which, when executed by the at least one server processor, further cause the server to: receive, via the second client device, one or more alterations to the EHR of the patient; transmit, from the second client device to the server, the one or more alterations; update, via the server processor, the patient information and the EHR based on the one or more alterations; regenerate, triggered by the one or more alterations to the patient information, via the at least one server processor, via the treatment adherence algorithm based at least on the portion of the patient information, the adherence probability of the patient. In an embodiment, the set of computer-executable server instructions which, when executed by the at least one server processor, further cause the server to: generate, via the second client device, a gating pop-up configured to alert a provider to validate the patient information within the EHR.

In an embodiment, the set of computer-executable server instructions which, when executed by the at least one server processor, further cause the server to: receive a full dataset; isolate a training dataset and a test dataset from the full dataset; split the training dataset into one or more training folds and a validation fold; train, over a plurality of trials, one or more models on each of the one or more training folds for each of one or more parameter configurations correlated to said one or more model, each of the one or more models configured to classify a target variable, wherein the target variable is an indicator of whether the patient will complete the treatment; validate, for each of the one or models for a given trial of the plurality of trials, a given model of the one or more models on the validation fold; compare a training score with a validation score, wherein the training score is based on the training of the one or more models over the one or more training folds, and wherein the validation score is based on the validating of the given model on the validation fold; record classifications scores for each of the one or more models, the classifications scores based on the training score and the validation score for each of the one or more models; select the treatment adherence algorithm from the one or more models, the treatment adherence algorithm having a top classification score; and retrain the treatment adherence algorithm on the full dataset. In a further embodiment, at least the training dataset comprises a plurality of variables, wherein each of the one or more models is configured to classify a target variable based on each of the plurality of variables. In yet a further embodiment, the plurality of variables comprises one or more continuous variables and one or more binary variables, wherein each of the one or more continuous variables is an integer or real number, and wherein each of the one or more binary variables is encoded as 1 if true, −1 if false, and 0 if missing.

In an embodiment, the plurality of variables comprises a normalized population density of a resident zip code, a normalized median income of a resident zip code, a normalized median home price of a resident zip code, a normalized number of total ICD-10 diagnoses, and a normalized number of ICD-10 diagnoses considered as psychiatric conditions. In an alternative embodiment, the plurality of variables comprises a normalized number of prior patients treated by a clinic with KIT, a normalized proportion of prior patients at the clinic that met a threshold for adherence to KIT, the patient's age at first infusion, the patient's BMI, and a normalized number of days patient had been associated with the clinic prior to their first KIT treatment. In a different embodiment, the plurality of variables comprises a normalized GAD7 composite score and a normalized PHQ9 composite score.

In an embodiment, the plurality of variables comprises a GAD-7 Item 1 Score, a GAD-7 Item 2 Score, a GAD-7 Item 3 Score, a GAD-7 Item 4 Score, a GAD-7 Item 5 Score, a GAD-7 Item 6 Score, a GAD-7 Item 7 Score, a PHQ-9 Item 1 Score, a PHQ-9 Item 2 Score, a PHQ-9 Item 3 Score, a PHQ-9 Item 4 Score, a PHQ-9 Item 5 Score, a PHQ-9 Item 6 Score, a PHQ-9 Item 7 Score, a PHQ-9 Item 8 Score, and a PHQ-9 Item 9 Score. In a further embodiment, the one or more continuous variables comprises sex, relationship status, completion status of intake form, mood disorder diagnosis, anxiety disorder diagnosis, attention disorder diagnosis, pre-visit status, and provider physician status.

These and other aspects, features, and advantages of the present invention will become more readily apparent from the following drawings and the detailed description of the preferred embodiments.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is an illustrative block diagram of a system based on a computer.

FIG. 2 is an illustration of a computing machine.

FIG. 3 is a workflow depicting an embodiment of the System.

FIGS. 4A-4C illustrate various embodiments of the patient interface.

FIG. 5 is an illustration of an embodiment of a provider-facing interface.

FIG. 6 is an illustration of an embodiment of a provider-facing interface.

FIG. 7 is an illustration of an embodiment of a provider-facing interface.

FIG. 8 is an illustration of an embodiment of a provider-facing interface.

FIG. 9 is an illustration of an embodiment of a provider-facing interface.

FIG. 10 is a workflow depicting an embodiment of the System.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

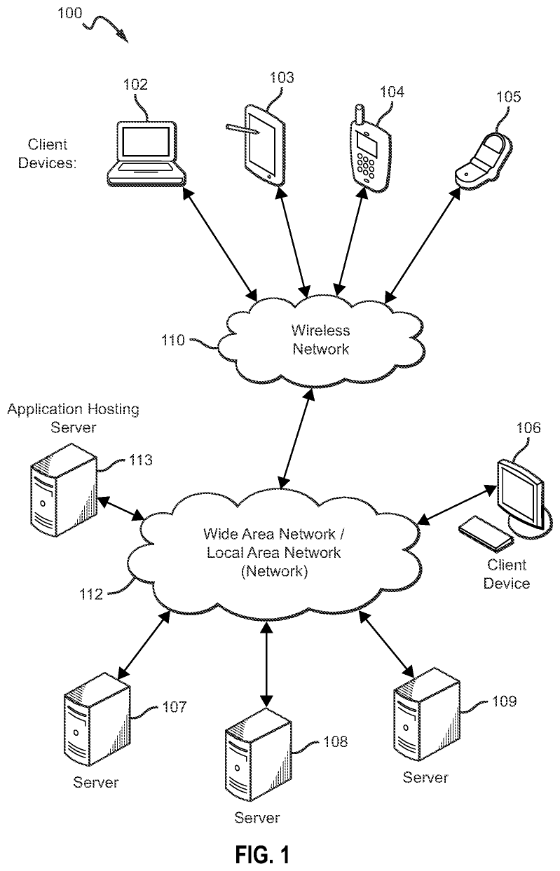

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

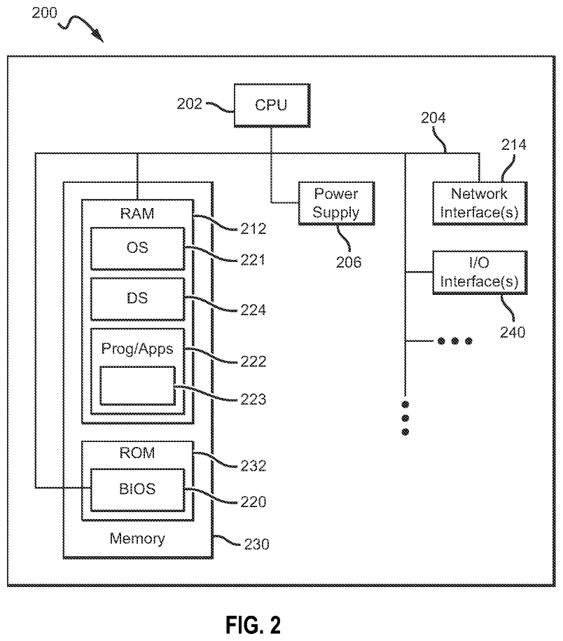

FIG. 2 illustrates a block diagram of an electronic device 200 that can implement one or more aspects of an apparatus, system and method for increasing mobile application user engagement (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

The system described herein may utilize said component/devices 240 (e.g., biometric sensors) to capture relevant information for the EHRs and/or predictive algorithm described below. Accordingly, the aforementioned data sources (e.g., biometric sensors) may be considered one or more external sources. The biometric sensors (also referred to as digital biomarker-capturing devices) may be configured to capture one or more digital biomarkers from the patient. In an embodiment, the biometric sensors may be separate from the client device. In another embodiment, the biometric sensors may be integrated within the client device. Accordingly, information collected by the biometric sensors may be imported to the EHR described below and/or the treatment adherence algorithm described below. Thus, in an embodiment, the treatment adherence prediction may be based, at least partially, on the data collected by biometric sensors. Yet further, data retrieved from any smart phone, wearable, or other device and/or components thereof (e.g., microphones, touchscreens, keyboards, mice, GPS components, cameras, light sensors, accelerometers, etc.) may be implemented in the predictive algorithm described below. In an embodiment, the utilization of biometric sensors and other peripherals may allow the system to readily update the predicted treatment adherence with data retrieved “on the fly.”

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MIMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

As noted above, embodiments of the present invention may relate to apparatuses, methods, and systems for integrating predictive models with an Electronic Health Record (“EHR”) platform. The embodiments may be referred to as the predictive model EHR integration system or simply, the “System.”

The System may utilize the computerized elements as described above and as illustrated in FIGS. 1-2. Accordingly, the System may include software elements configured to execute the features described herein.

A predictive algorithm may be central to the System, wherein the algorithm may employ evidence-based medicine as informed by large samples of patients and valid statistical analysis. In such an embodiment, the System, via the algorithm, may calculate and/or distribute an easily-interpreted probability estimate. Such an estimate may be used to create a software feature in any suitable electronic health record (EHR) platform. In an embodiment, a display of the calculated probability is embedded into the clinical workflow, such that a provider may view the calculated probability within the patient chart when such a provider is examining which treatments may be successful for said patient. Traditional EHRs do not natively include such calculated probabilities. Accordingly, such traditional EHRs cause a fragmented user experience requiring double data entry, excessive manual clicking to calculate numbers, navigation through third-party tabs, windows, and popups. As described herein, the improved platform provides succinct integration of the EHR platform and the calculated adherence probability. Yet further, the improved System described herein may be configured to identify and manage certain fields that may be empty within a patient record, but are required for probabilities to be calculated.

The estimate determined herein may enable clinicians to make a data-driven decision on whether to offer a treatment (for example, including administration of ketamine) to a particular patient. As such, the estimate may be displayed alongside other estimates for additional treatment target outcomes provided by alternative algorithms, such that clinicians are enabled to select from a number of treatment options as informed by these estimates. In an embodiment, various probability estimates for various treatments may be presented on the interface. As a non-limiting example the platform and interfaces thereof may include a dashboard showing all the possible treatments and the likelihood of success for each. Accordingly, such a dashboard would assist clinicians in deciding which treatment would be most likely to have high adherence and, thus, best patient outcome.

In an embodiment, the probability prediction may be displayed alongside various other data (e.g., patient reported outcomes, patient intake information) and corresponding tools adapted to initiate treatment (e.g., scheduling and clinical notes functionality).

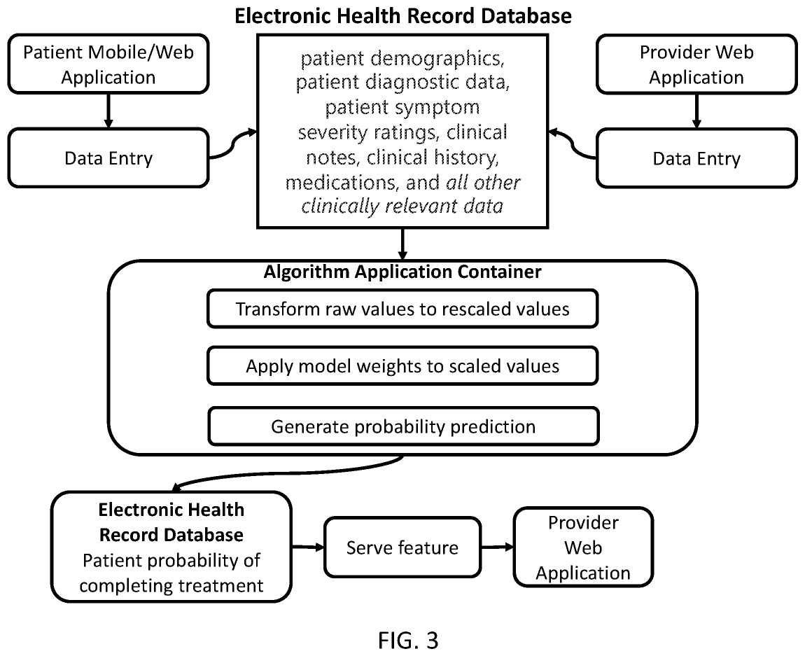

Referring to FIG. 3, the System may comprise a patient mobile/web application. The patient mobile/web application may be a software element executable and/or displayed on a patient's device (for example, a smart phone, computer, or other digital device). Such a patient device may include one or more peripherals enabling input and retrieval of data. In an embodiment wherein the patient device is a smart phone, a patient interface may be generated and displayed, wherein interaction with the patient interface via a touch screen enables data entry. Accordingly, as shown in FIG. 3, the patient mobile/web application may be in communication with an EHR database, for example, to facilitate data entry from the patient mobile/web application to the EHR database. In an embodiment, the EHR platform is configured to extract information (e.g., metadata) from a user's device (e.g., smart phone or computerized wearable). As a non-limiting example, the EHR platform may be configured for “digital phenotyping,” which is the use of digital data from devices (e.g., accelerometer data to inform location, social activity, or sleep habits). Such information may be highly relevant clinically to help “phenotype” the patient's psychiatric condition, and thus plays a role in predicting how a patient might adhere to treatment.

The EHR database may be stored in a server storage device as described above and illustrated in at least FIGS. 1-2. As a non-limiting example, the patient interface may be configured to capture patient data through various features including, but not limited to, written journaling, patient reported outcomes (e.g., validated mental health rating scales), secure messaging, verbal journaling (e.g., microphone captured audio), and image upload.

The System may also include a provider web application. The provider web application may include an interface adapted to generate and/or display on a provider's device (for example, a smart phone, computer, or other digital device). In an embodiment, the provider web application is configured specifically for mental health clinicians, such that the workflows, user interface, and user experience are tailored and fit for said purpose. As a non-limiting example, each data field within the patient chart is relevant to a mental health clinician versus a generic EHR that may ask a number of questions relevant for a primary care physician (PCP), but not a psychiatrist. Accordingly, the user experience is improved for the provider. The provider may follow the workflows, which may be automated and support a more efficient experience. Similarly, embedding the algorithm predictions directly into the EHR and clinician workflow may render the algorithm's prediction most actionable, most helpful, and, thus, most effective.

The provider interface may be configured to receive data input from one or more providers. Thus, the provider interface may enable data entry to the EHR database. In an embodiment, the provider interface includes one or more permissions enabling access to administrators and/or approved providers (for example, as a function of the provider's correlation to a particular patient or class of patients). As a non-limiting example, the provider interface may be configured to capture data through various features including, but not limited to, an appointment scheduler, billing and invoicing modules, and telehealth encounters.

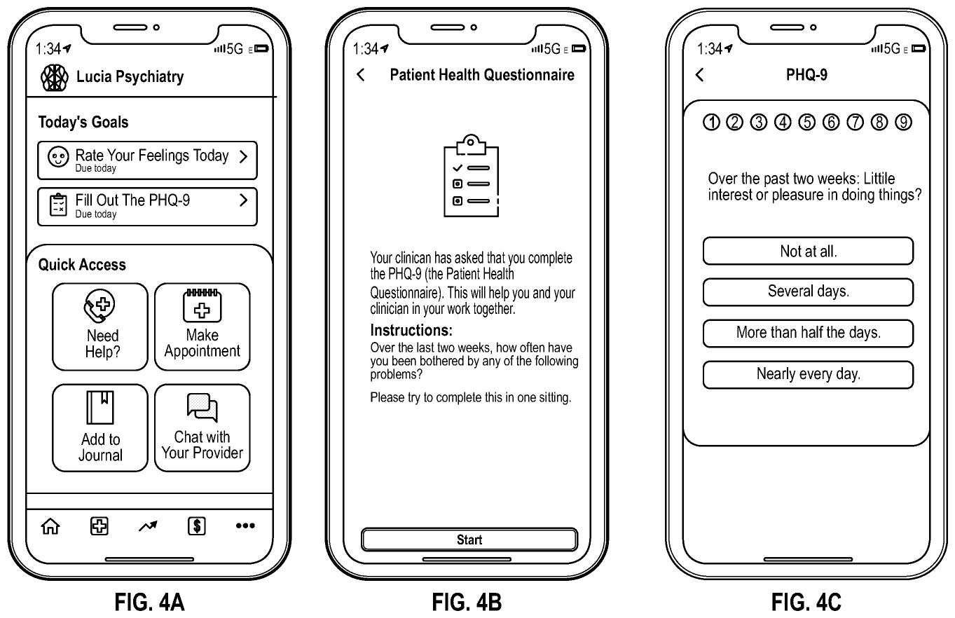

FIGS. 4A-4C illustrate various embodiments of the patient interface. In an embodiment, the patient interface is configured to increase user engagement. Such an improvement to user engagement may be manifested by the collection of patient reported outcomes in addition to administrative workflows (e.g., billing, scheduling, and payments). In such a sense, the system may be an improvement because the platform is not merely an administrative portal, but instead a patient-facing interface, allowing the patient to actively engage in their own care. Accordingly, the patient may track their own outcomes. Such improved tracking means are not available in traditional EHR platforms or related patient-facing interfaces.

For example, FIG. 4A may illustrate a patient-facing interface, such as an initial patient interface and/or a patient home screen, wherein the patient home screen provides various options to the patient. For example, the patient home screen may provide a selectable patient health questionnaire (i.e., the PHQ-9). FIG. 4B may illustrate a patient-facing interface, such as an instructional screen of the patient health questionnaire. FIG. 4C may illustrate a patient-facing interface, such as an example of a question within the patient health questionnaire. The patient health questionnaire may provide one or more selectable answers correlating to each of the presented questions. The patient health questionnaire may be configured to extract data from the patient, wherein such data may be stored in the EHR database (for example, to be retrieved and analyzed in view of the algorithm).

The patient's data and the provider's data may be stored within the EHR database. The EHR database may include patient demographics, patient diagnostic data, patient symptom severity ratings, clinical notes, clinical history, medications, and all other clinically relevant data. The EHR database may be structured wherein each of the data points is correlated to a particular patient and/or provider. For example, data entry originating from the patient mobile/web application may be tagged to correlate said information with a particular patient. Similarly, data entry originating from the provider web application may be tagged to correlate said information with a particular provider. Thus, the database may be appended as data is received from the patient mobile/web application and/or provider web application. In an embodiment, the System described herein may utilize the categories of input data described in the feature list below. However, in further embodiments, the System may be configured to receive and analyze additional input data points. As a non-limiting example, such additional input data points may be provided electronically or verbally (for example, via a microphone embedded within the user's device) by the patient and may be entered into the electronic health record corresponding to said patient. As another non-limiting example, additional input data points may be measured from passive-sensing (e.g., a patient may connect their smart device or wearable to the System, wherein the smart device or wearable may upload activity history and utilize such data as an additional input). Moreover, as described in further detail below, the System is operable if individual data fields are missing. The System described herein may be adapted to be agnostic to all data intakes and/or may be configured for use with a universal data intake. The System may include a dedicated intake module configured to perform intake within the platform used to collect some of the input data.

Referring to the patient interface and/or patient mobile/web application, the System is configured to generate predictions based on standard intake data. For example, multiple data points may be collected during a standard intake examination. Such data points may be combined and converted into a single probability estimate. In an embodiment, the probability estimate ranges from 0 to 1 (non-inclusive), however, the probability estimates may be represented in percentages, fractions, ratios, or other suitable forms. The probability estimate may indicate the likelihood that an individual patient will complete a predetermined treatment threshold. The probability estimate may be accompanied with a reference to background documentation and/or explanations of the research supporting the technical performance of the algorithm. The predetermined treatment threshold may be determined experimentally and/or may be set by a System administrator. However, the predetermined treatment threshold may be modified and/or otherwise altered during System operation and/or between instances of use. Further, the predetermined treatment threshold may be specific to a particular type of treatment and/or a particular patient or class of patients. As a non-limiting example, the predetermined treatment threshold may include at least 4 infusions of IV ketamine within 28 days of the intake examination. However, the predetermined treatment threshold may be any measure of the completeness of a particular treatment regimen. Thus, as an example, the predetermined treatment threshold may be represented as a sufficient dosage or duration of treatment relative to the preferred dosage or duration of treatment. In an alternate embodiment, the predetermined treatment threshold may be a range of values (for example, 4-8 infusions of IV ketamine within 28 days).

Predictions may be generated based on standard intake data and/or additional types of data. For example, such additional types of data include, but are not limited to, intake data, clinical notes, patient reported outcomes, demographics, psychiatric history, medical history, social history, family history, medication history, diagnoses, and allergies. In various embodiments, one or more of the aforementioned additional types of data are included in a standard intake, while some others are generated during a clinical encounter rather than the intake process. As a non-limiting example, during a routine visit the clinician could ask the patient about their family history and input that data, rather than having that information already input during the intake.

Referring to FIG. 3, the underlying algorithm may be utilized to generate probability predictions that may be implemented into electronic health record software. The data inputs for the algorithm may be entered into a patient mobile or web application and/or a provider web application. In one embodiment, the EHR database may be stored in a proprietary and/or internal server. However, in further embodiments, the algorithm and/or other System components may be utilized in conjunction with and/or may be in electronic informatic communication with any suitable EHR platform. For example, the System may include one or more Application Programing Interfaces (“API”), wherein the API is configured to implement transmission of requests and responses between the EHR database internal to the System and/or an external EHR database. Therefore, in such a non-limiting example, third-party EHR platforms, digital health suites, or other medical networks may utilize the generated prediction probability. A probability for treatment adherence is valuable outside of the clinical encounter between patient and psychiatrist. As a non-limiting example, the probabilities can be surfaced and used to assess quality of psychiatric care (e.g., does a psychiatrist correctly choose treatments that have optimal likelihood of adherence) and reported to medical societies, healthcare delivery systems, or payers. Such probabilities can be very useful at the population level, not just the individual clinician level. Such probabilities may be used to influence treatment guidelines (e.g., with a medical society or academic research partners), help pharmaceutical companies with their medical affairs initiatives and pipeline planning projects, help payers determine treatment algorithms and reimbursement pathways, and more.

As a non-limiting example, the platform, algorithm, and adherence probability described herein may be adapted for importing IV ketamine treatment adherence predictive algorithms into the EHR. However, the methods and systems described herein are not limited to the instance of IV ketamine treatment. Accordingly, the methods and systems described herein may be utilized for any and all mental health treatments, enabling adherence predictive algorithms for a wide variety of health conditions and treatments to integrate into the EHR. As a non-limiting example, such health conditions and treatments may include rapid-acting antidepressants or interventional psychiatric treatments.

In an embodiment, a software container comprises the computer-executable instructions to convert the raw data stored in the EHR database to the patient's estimated probability of treatment adherence. In an aspect of the System, this conversion may comprise the following steps: each raw data point may be converted into a rescaled value; according to the formula provided by the trained algorithm, a multiplier or “weight” may be assigned to each input data point to calculate the change in probability associated with that specific input; and all inputs may be summed to generate the final probability prediction. However, the conversion may comprise any number and/or combination of suitable steps to extract a prediction probability from the data provided by the patient and/or provider. The aforementioned conversion is described in further detail below.

The data utilized by the algorithm may be retrieved from clinical measures or processes known to persons of ordinary skill in the art. However, the data may be retrieved from any suitable measures or processes. As a non-limiting example, such data retrieval may include, but is not limited to, Quick Inventory of Depressive Symptomatology (QIDS-SR16, hereafter referred to as “QIDS”); Generalized Anxiety Disorder-7 (“GAD-7”); Psychiatric evaluation for ICD-10 diagnoses of mood and anxiety disorders; and/or demographic assessment of age, race, ethnicity, and/or sex. Accordingly, as to decrease required deviation from standard clinical procedures, each measure may be a standard assessment that a clinician may perform when conducting an intake evaluation for treatment of the target condition (for example, depression). Therefore, the data points generated by these measures and used by the algorithm may include, but are not limited to, the QIDS total score; the GAD-7 total score; for demographics, 4 variables with the default value of 0 (for example, values are 1 if patient is: 1. male sex, 2. missing sex data, 3. Caucasian, 4. not Caucasian); age; for ICD-10 diagnoses, 4 variables with the default value of 0 (for example, values are 1 if patient has a diagnosis of: 1. major depression, 2. an anxiety disorder other than post-traumatic stress disorder [PTSD], 3. PTSD, 4. bipolar disorder); and a count of the number of different ICD-10 diagnoses a patient has. However, the aforementioned measures should not be viewed as limiting, the metrics analyzed by the algorithm may include any number or combination of variables set to any suitable default values or thresholds.

In an embodiment, the algorithm and/or predictive aspects of the System may be bolstered by datasets of patients being administered the target treatment (for example, initiated IV ketamine treatment). Accordingly, the EHR database may be utilized to track compliance and/or adherence with the particular target treatment. Thus, EHR data of a class of patients (for example, each prescribed the same treatment) may be utilized in training the algorithm (for example, modifying models, variables, weights, etc.).

In further embodiments, data retrieved from intake may include the patient's history of motion sickness and/or vertigo (for example, as a predictor of noxious side-effects during KIT). In an embodiment, historical variables may include those computed from the provider entity/clinic-side data, such as indicators of the clinic's historical experience with delivery of intravenous ketamine treatments or other relevant treatments. Accordingly, one or more data points may be correlated to the clinic(s) offering the relevant treatment. Thus, the algorithm, and resulting predicted treatment adherence, may be a function of the patient's clinic's history and other characteristics. As a non-limiting example, if a particular patient is enrolling in a particular clinic that has shown substantial treatment adherence, the algorithm may provide a greater probability to said patient's success versus a patient enrolled in a subpar clinic. However, the data retrieved from intake may include any data points, variables, and/or may illuminate any such connections between intake data and the underlying treatment.

As described above, each treatment may include a corresponding target variable (for example, a predetermined treatment threshold). For the purposes of IV ketamine infusions, the desired target variable for the corresponding dataset may be an indicator of whether a patient completed the prescribed minimum initial course (induction) of IV ketamine infusions. In such an embodiment, the minimum initial course is defined as completing at least 4 infusions within 28 days from the intake evaluation. However, the target variable (predetermined treatment threshold) may be any measure of completeness, such as full course completion, completion of an initial course, or completion of a desired percentage or stage of the treatment regimen. Thus, for the purpose of training statistical models to predict said target variable, patients who completed the minimum course may be assigned a value of 1 and the remaining patients may be assigned a value of 0. However, the patients may be assigned any value between 0 and 1 correlating to their associated treatment completion status. As a further non-limiting example, alternative models may be configured to predict a number outside the range of 0 to 1. Moreover, any suitable statistical analysis metrics and methods may be utilized.

As an example, the mathematical relationship between the inputs and the estimated probability of IV ketamine adherence may be represented, in part, by a probability function p(x), where p(x) is defined as the probability that a given patient will complete at least 4 sessions of IV ketamine treatment. However, the p(x) function may be altered and/or otherwise modified to determine the probability of completion of any portion or milestone of any treatment and/or drug regimen. Thus, the p(x) function should not be viewed as limited to the example of IV ketamine treatment. The algorithm may utilize:

$$p ( x ) = \frac{1}{1 + {e}^{- ( f )}}$$

wherein f is a linear formula defined by one constant intercept and 37 multiplicative coefficients applied to 37 measured variables as such:

f=β₀+β₁x₁+β₂x₂+β₃x₃ . . . +β₃₇x₃₇

wherein β₀ is a constant value and β_{1 . . . 13} are coefficients.

The 37 variables in f may be defined as such:

| | |
|---|---|
| x₁ | Normalized population density of patient's resident zip code |
| x₂ | Normalized median income of patient's resident zip code |
| x₃ | Normalized median home of patient's resident zip code |
| x₄ | Normalized number of total ICD-10 diagnoses |
| x₅ | Normalized number of ICD-10 diagnoses considered as psychiatric conditions |
| x₆ | Normalized number of patients clinic has previously treated with KIT |
| x₇ | Normalized proportion of prior patients at the clinic that met the threshold for |
| | adherence to KIT |
| x₈ | Normalized patient's age at first infusion |
| x₉ | Normalized patient's BMI |
| x₁₀ | Normalized number of days patient had been associated with the clinic prior to |
| | their first KIT treatment |
| x₁₁ | Normalized GAD7 Composite Score |
| x₁₂ | Normalized PHQ9 Composite Score |
| x₁₃ | GAD-7 Item 1 Score |
| x₁₄ | GAD-7 Item 2 Score |
| x₁₅ | GAD-7 Item 3 Score |
| x₁₆ | GAD-7 Item 4 Score |
| x₁₇ | GAD-7 Item 5 Score |
| x₁₈ | GAD-7 Item 6 Score |
| x₁₉ | GAD-7 Item 7 Score |
| x₂₀ | PHQ-9 Item 1 Score |
| x₂₁ | PHQ-9 Item 2 Score |
| x₂₂ | PHQ-9 Item 3 Score |
| x₂₃ | PHQ-9 Item 4 Score |
| x₂₄ | PHQ-9 Item 5 Score |
| x₂₅ | PHQ-9 Item 6 Score |
| x₂₆ | PHQ-9 Item 7 Score |
| x₂₇ | PHQ-9 Item 8 Score |
| x₂₈ | PHQ-9 Item 9 Score |
| x₂₉ | 1 if patient is male, 0 otherwise |
| x₃₀ | 1 if patient is in relationship, 0 otherwise |
| x₃₁ | 1 if patient filled out an intake form for the clinic, 0 otherwise |
| x₃₂ | 1 if patient is diagnosed with a mood disorder, 0 otherwise |
| x₃₃ | 1 if patient is diagnosed with an anxiety disorder, 0 otherwise |
| x₃₄ | 1 if patient is diagnosed with an attention disorder, 0 otherwise |
| x₃₅ | 1 if patient had a visit at the clinic prior to their first KIT treatment, 0 otherwise |
| x₃₆ | 1 if patient's provider is a physician, 0 otherwise |
| x₃₇ | 1 if patient's provider has an advanced nurse or nurse practitioner credential 0 |
| | otherwise |

However, the 37 variables in f may be defined as any suitable measures or metrics derived from patient intake data. Additionally, f may include any suitable number and/or combination of variables.

As a non-limiting example, the normalization formula for numeric variables may be defined as:

xᵢ=(x_{raw}−xₘᵢₙ)÷(xₘₐₓ−xₘᵢₙ)

wherein terms may be defined as:
xᵢ=Value of x input to algorithm
x_{raw}=Original measured value of x
xₘₐₓ=Sample maximum value of x
xₘᵢₙ=Sample minimum value of x.

In an embodiment, the normalization approach is dependent on whether the variable is a continuous variable or a binary variable. In an embodiment, for continuous variables (i.e., variables whose values are integers or real numbers and not a simple true or false), outliers values may be removed, for example, using a kernel density estimation approach. Accordingly, the probability density function of the variable may be estimated, any variables with a probability density lower than a predetermined percentage of the maximum density may be removed. A power transformation may be applied to reshape the distribution of values to be approximately Gaussian (xₜ=transform(x)). In an embodiment, missing variables may be ignored during the reshaping of distributions. The values may be scaled using a z-transformation, for example: x_{z}=(xₜ-mean(xₜ))/st_dev(xₜ). Missing values may be ignored during the z-transformation. Missing values may be “imputed,” wherein a value of 0 may be inserted for all unknown values or values that were removed as outliers earlier. In an embodiment, for binary variables, variables may be coded as 1 if true, −1 if false, and/or 0 if missing. Although the above means of normalization may be utilized for one or more of the models described herein, some classifiers may utilize additional normalization steps, which are contemplated below.

In alternate embodiments, different data points may be used as inputs to the algorithm, (either by removing a number of inputs, adding new inputs, or replacing a number of inputs with others). As a non-limiting example, the PHQ-9 depression measurement may be replaced and/or supplemented with a different measurement metric, such as QIDS. Accordingly, modification of the inputs may alter the algorithm. The algorithm may assign different weights to existing inputs if other inputs were added or were removed, and any replacement inputs may also have a different weight assigned to them.

For the purposes of this disclosure, a classifier may be a statistical model, wherein said model may be created with the purpose of accurately correlating a given data input (e.g., EHR data) with a class (“completed”). In an embodiment, the classification model may be constructed using supervised training, wherein at least one input may be comprised of at least one label, and wherein the at least one label may identify a known class. Said labels may be utilized to train the classifier. In a further embodiment, the classifier may be able to actively learn how to correctly assign a class, wherein the assignment of the class may be based upon labeled training data. In an alternative embodiment, the classifier may then be used to determine the classes of unlabeled input for which the class is unknown. In effect, the classifier may be adapted to

In another alternate embodiment, a different type of classifier may be implemented. The classifier may be a “linear” model, wherein the form of the algorithm is restricted to a linear formula. However, the classifier may be nonlinear in nature, such as decision tree models or K-Nearest Neighbors models. The classifier may also be characterized as an “ensemble” model, wherein the function of the classifier may be generated by a combination of multiple individual classifiers. Such an ensemble model may not be sufficiently described in a single formula as displayed above. “Ensemble” may refer to any type of model where multiple individual models (aka “base estimators”) contribute to the predictions. As a non-limiting example, two distinct types of models (e.g., logistic regression and naive Bayes) could be developed using the same features dataset and target. In one such instance, the first model type predicts a positive class probability of 0.80, the second model type predicts a positive class probability of 0.60, and the ensemble algorithm may average both probabilities to produce a final probability of 0.70. As a further non-limiting example, the System may be equipped to utilize algorithms commonly implemented as ensembles in standard machine learning software packages (e.g., a random forest model, which is an ensemble of decision trees, where the predictions of tens, hundreds, or even thousands of decision trees can be averaged to arrive at the final prediction).

The classifier may also be characterized as a “black box” model, wherein the transformations applied to the data inputs may occur in a process comprising multiple individual steps that are dynamic and interdependent. Similarly, such a black box model may not be sufficiently described in a single formula as displayed above. Further, such a black box model may be applied through application of a trained model directly in software. In an alternate embodiment, the System may include “stacked” implementations of algorithms, wherein the output(s) of one algorithm may be fed as inputs to one or more additional algorithms to produce the final predictions. Additional classifiers may include: Bayesian linear models (which may be similar to linear models, but estimate the uncertainty in the relationships between model variables and outcomes); hierarchical models (which model parameters may have different values based on their relationships with other variables. For example, the “weight” for PHQ-9 scores may be different for patients who are treated by a physician vs. patients who are treated by a provider with an advanced nursing credential); naive Bayes classifiers (which leverage Bayes Theorem to estimate how likely a set of variables is to appear in one “class” vs. another); and/or kernel-based methods, including but not limited to Support Vector Machines, Relevance Vector Machines, and Gaussian Process Classification, which use linear methods on data that is transformed to a high-dimensional, non-linear space.

In further embodiments, to improve the algorithm performance, additional data points may be included to provide new inputs to the model. There may be additional explanatory variables that, when included, aid in error correction and/or improve overall classification performance. The present disclosure contemplates the possibility that derivations of existing input data may improve performance when added to the feature set. For example, additional new variables computed from diagnostic data or multiplicative combinations of features may add additional explanatory power to predictive models. Additionally, data integrated with smartphones and/or biotech assessments (e.g., brain electrical activity measures) may be utilized.

Such additional inputs may be derivatives of the included inputs (for example, the product of two or more inputs or the squared value of a single input). Alternatively, such additional inputs may be derived from new measures and data points not included in the initial inputs. In one embodiment, inputs may be removed to improve performance of the algorithm.

Further, as described above, any suitable classifier may be implemented.

Turning back to FIG. 3, the user of this System may be a clinician who provides IV ketamine treatment and is a user of the EHR platform. However, the System may be configured for use with any suitable class or type of treatment. The EHR database may store patient and/or provider information as a “patient's chart.” The patient's chart may be a viewable chart comprising features embedded within the chart adapted for data capture and/or for providing access to the algorithm application container. The patient's chart may include a presentation and/or an interactive view of a selected patient's data. Such data may include clinical information, rating scales, clinical history, and other relevant metrics. All necessary inputs may be entered into and stored within the patient's chart, including, but not limited to, demographic variables, diagnosis variables, and symptom severity ratings. As described earlier, these inputs may be entered by the patient in the patient mobile application or patient web application and/or entered by the clinician in the provider web application. In an embodiment, any information not provided by the patient via the patient interface may be entered by the provider via the provider interface. As a non-limiting example, a clinician may enter data via the provider interface if the patient has failed to enter such data through the patient interface. In an embodiment, the System and/or algorithm may be configured to include different weights to information extracted from the patient interface versus information extracted from the provider interface. However, in another embodiment, no favorability may exist as to the source of information. In some aspects, certain information (i.e., diagnosis) provided by the patient may be superseded by the clinician if the clinician opts for the System to do so. As a non-limiting example, a patient may claim to have a diagnosis of “bipolar disorder” and recites as such in the patient interface, yet the clinician may remove said diagnosis upon further examination. In an embodiment, the algorithm is dynamic and the predicted probability may be re-calculated upon changes in relevant parameters in the patient chart. As a non-limiting example, the System may be configured to generate and display a gating pop-up (or a similar alert) that prompts the clinician to verify that relevant fields are correct before generating the probability for the first time. These extracted data points and values may be stored in an EHR database, wherein the EHR database may be an object-relational database (for example, a PostgreSQL database). However, the EHR database may be any suitable database structure or format. The logic aspect of the algorithm may reside in an application container, which may be a self-contained module containing all software aspects and computer-executable instructions required to ingest the inputs from the EHR database into the algorithm and produce the predicted probability. The algorithm may operate on a server. The server may be configured to execute the logic contained in the container when the function held in the container is called by some action. This action may be called from within the System and/or software (for example, upon a user's click in the patient and/or provider interface) or an additional process. As a non-limiting example, the System may include a routine adapted to auto-generate a prediction when a certain set or all the input data is available. The algorithm may also be configured to generate a predicted probability as a function of a schedule. For example, a schedule may be configured to update all model predictions across the EHR database every 10 minutes. Moreover, the advantage of the container may be that the System does not require a dedicated server to be available at all times as the logic may only be actuated when needed.

The System may include one or more triggers, causing the System to run. In an embodiment, the System may be configured to run the algorithm's logic based on a predefined event (for example, when all necessary inputs have been provided in the database) or upon request by the clinician. Upon probability prediction generation, the probability value may be displayed in the patient's chart in the provider web application and may be made available within seconds of running the algorithm. In an embodiment where the algorithm is actuated upon request of a clinician, a signal may be generated in a patient's chart signifying that the patient has a prediction available. In such an embodiment, the patient's chart may include a selectable button to make the prediction visible. Alternatively, predictions may be auto-served for all patients when all input data points are available (e.g., at the level of provider or the level of clinic). A System administrator may enable the aforementioned feature for a certain set of providers or clinics. However, such a feature may be enabled for all users.

In a further embodiment, the System may present the probability of treatment adherence in comparison to alternate treatments. For example, a given clinician providing IV ketamine treatment may also be able to offer alternative treatments, such as transcranial magnetic stimulation, antidepressant treatment, or electroconvulsive therapy. Thus, if a given new patient has a high or low probability of adhering to the prescribed IV ketamine treatment regimen, the clinician may utilize such information when deciding which treatment to offer the patient. In yet another embodiment, the System may be configured to generate probability predictions for a plurality of treatment types. In such an embodiment, the algorithm may be modified to evaluate a plurality of treatment types and/or the System may include a plurality of algorithms, each of the algorithms configured to generate a prediction for a corresponding treatment type. Therefore, the System, and specifically the System's integration within an EHR platform, may enable selection of a treatment based on a plurality of predictions. In an embodiment, after a clinician decides which treatment to administer, then they just follow the typical workflows in the software that would correlate to that treatment (e.g., writing a clinical note using the template for that specific intervention, prescribing a medication using the e-prescribing functionality, referring out to a third party clinician and sending relevant parts of the medical record from the software).

Further, the System may be configured for updating the model's predictions in “real-time” as a patient progresses through the course of treatment. For example, an algorithm trained on the set of patients who have completed a particular milestone (for example, at least 1 ketamine infusion) may be utilized to illuminate evaluation at that point in the course of treatment (for example, as compared to the predictions served after the patient's initial evaluation).

The algorithm described herein may be configured to make accurate predictions on out-of-sample data. Accordingly, the algorithm may be adapted to utilize a machine learning framework in conjunction with readily acquired standard patient intake data to predict likelihood of adherence to a prescribed regimen of IV ketamine treatment for a particular patient.

The algorithm may include transforming the raw values of clinical measures (for example, QIDS, GAD-7) into rescaled values. This step, often referred to as “Scaling” or “Normalizing” the data, may be taken both when fitting the model to training data in the data experimentation workflow outlined in FIG. 3 and when the model is implemented in software for making predictions on new samples. This step may improve both the data experimentation workflow and the software implementation. Accordingly, normalizing the data increases accuracy of predictions because, for some inputs, the algorithm's weights may be either much too small or too large for the unscaled data.

Once a probability prediction is generated by the algorithm, via the algorithm application container, the probability prediction may be transmitted to the EHR database. Like the information originating from the patient mobile/web application and/or the provider web application, the probability prediction may be tagged or otherwise correlated to the particular patient.

The System may be further configured to transmit the patient probability of completing a particular treatment to the provider web application. Thus, the probability prediction may be retrieved by the provider via the provider web application. In such an embodiment, access to the provider web application may be restricted based on permissions set by an administrator or automatically by the System. For example, a particular provider may be permitted access to data correlated to that particular provider's patients.

The invention of the present disclosure may be automated as to consider multiple simultaneous data points when making clinical decisions. After data collection, the algorithm, and System generally, may automatically generate the desired output and feed that output back to the clinician. In such an embodiment, clinicians receive valuable intelligence from the prediction probability without completing additional work, saving them valuable time and effort. The integration of the System into an EHR platform may provide clinicians with a validated, predictive clinical decision support tool to guide treatment decision-making.

The algorithm described herein may be executed and/or used in connection with any suitable machine learning, artificial intelligence, and/or neural network methods. For example, the machine learning models may be one or more classifiers and/or neural networks. However, any types of models may be utilized, including regression models, reinforcement learning models, vector machines, clustering models, decision trees, random forest models, Bayesian models, and/or Gaussian mixture models. In addition to machine learning models, any suitable statistical models and/or rule-based models may be used.

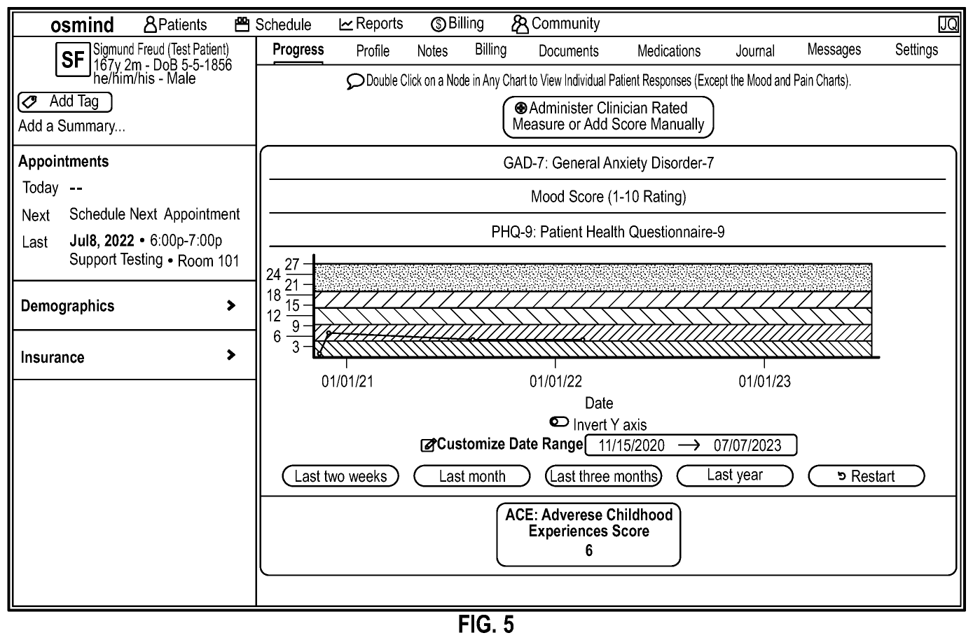

FIG. 5 is an illustration of an embodiment of a provider-facing interface. As shown in FIG. 5, the provider-facing interface may include information regarding the patient and the patient's diagnosis and/or treatment progress, wherein said information regarding the patient may include, patient demographics and/or patient insurance information. In an embodiment, the provider-facing interface may include data, wherein the data comprises at least one of a GAD-7 Score, a Mood Score, a PHQ-9 Score, and an Adverse Childhood Experience (ACE) Score. In an alternative embodiment, the aforementioned data may be displayed in a pictorial illustration (i.e., a graph), wherein said illustration may allow for the data to be selectively displayed. Moreover, the pictorial illustration may allow for customization of variables on at least one illustration axis. In a further embodiment, the provider-facing interface may include patient appointment data that displays at least one of a next scheduled appointment and a previous appointment, wherein the next scheduled appointment and the previous appointment may include a date, a time, a reason for the appointment, and/or a location of the appointment.

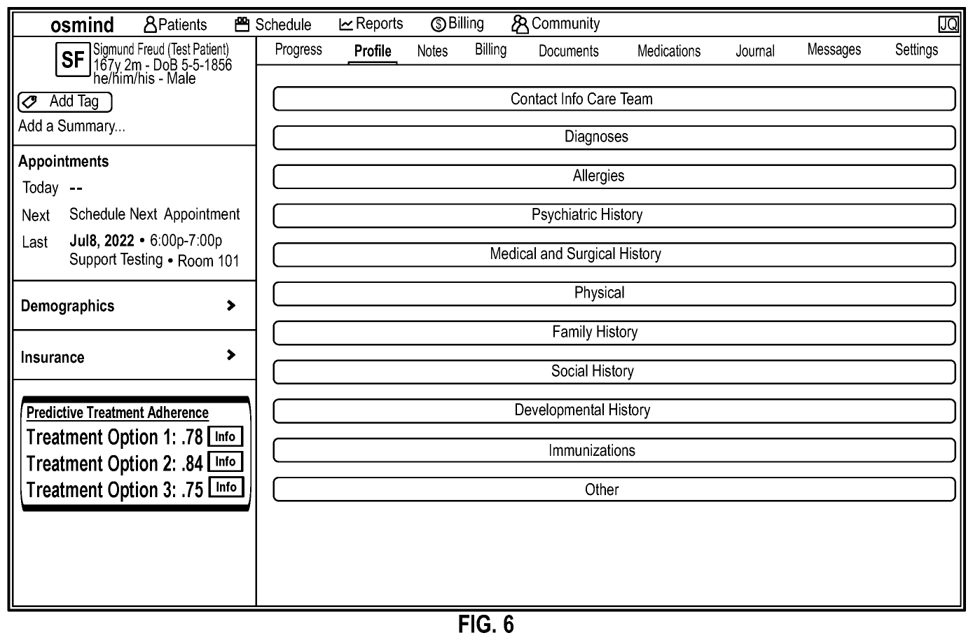

FIG. 6 is an illustration of an embodiment of a provider-facing interface. As shown in FIG. 6, the provider-facing interface may include a patient profile, wherein said profile includes information regarding said patient as well as personal patient information. In an embodiment, the personal patient information may include at least one of contact information, diagnoses, allergies, psychiatric history, medical and/or surgical history, physical patient information (e.g., height, weight, BMI, etc.), family history, social history, developmental history, immunizations, and other information. However, the personal patient information encompassing the patient profile may include any suitable and relevant information alternative. The provider-facing interface may include a predictive treatment adherence module configured to display the predicted treatment adherence probability of one or more potential treatments. For example, the predictive treatment adherence module may include treatments, each treatment's corresponding probability as dictated by the algorithm described herein, and an “info” button (or an equivalent features) enabling a user to review additional detail about the treatment and/or the probability. As a non-limiting example, the “info” button, when actuated, may inform the user of the treatment regimen details (e.g., duration, medication schedule, etc.), the model/algorithm used to determine the probability, and the factors evaluated by said model/algorithm.

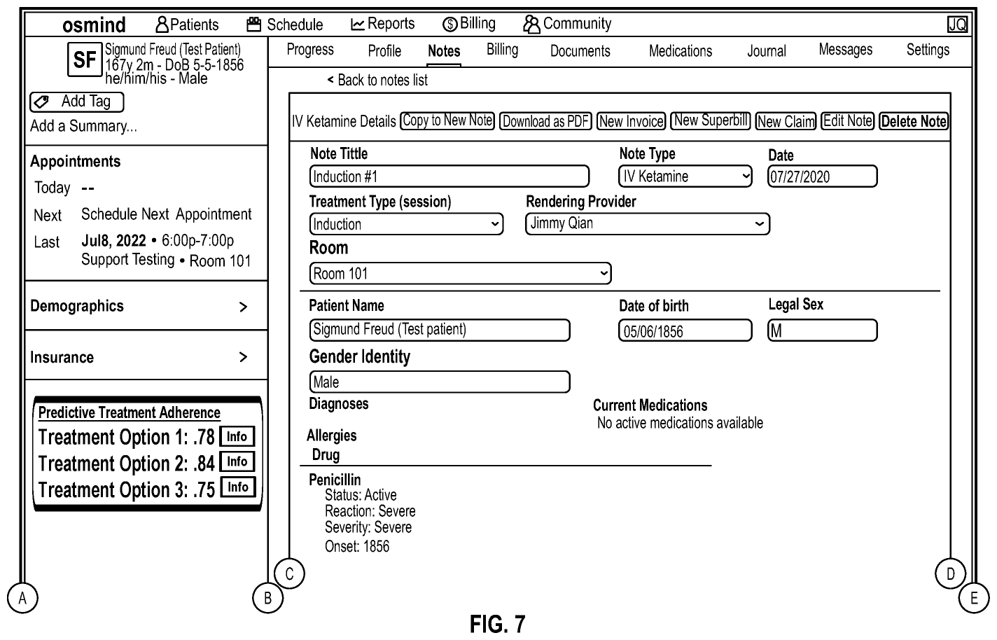

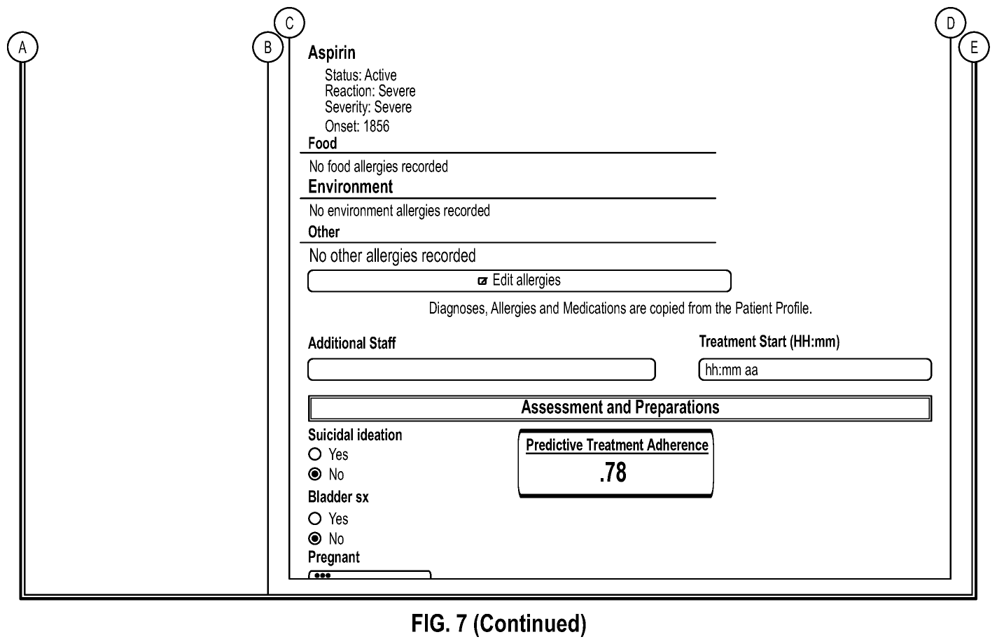

FIG. 7 is an illustration of an embodiment of a provider-facing interface. As shown in FIG. 7, the notes interface may include a representation of the predicted treatment adherence for the selected treatment. A clinical note page may correlate to one or more treatments, for example, as shown in FIG. 7, IV ketamine treatment. The clinical note page may include a feature depicting the predicted treatment adherence. Such a feature may be informed by the treatment adherence predictive algorithm described herein. In a further embodiment, each clinical note page and/or treatment having a correlated treatment adherence predictive algorithm may also include a feature depicting the respective predicted treatment adherence. In an alternative embodiment, the notes interface may include information regarding the patient and/or personal patient information. Moreover, the notes interface may allow for a healthcare provider to take notes regarding the patient. The note interface may provide a plurality of input sections, wherein said sections may include at least one of a note title, a note type, a date, a treatment type, a rendering provider, and a location. Further, the notes interface may include a plurality of tabs, allowing the healthcare provider to customize the notes regarding the patient. Said tabs may include sections to copy the notes to a new note, download the notes as a PDF, create a new invoice, create a new superbill, create a new claim, edit the notes, and/or delete the notes.

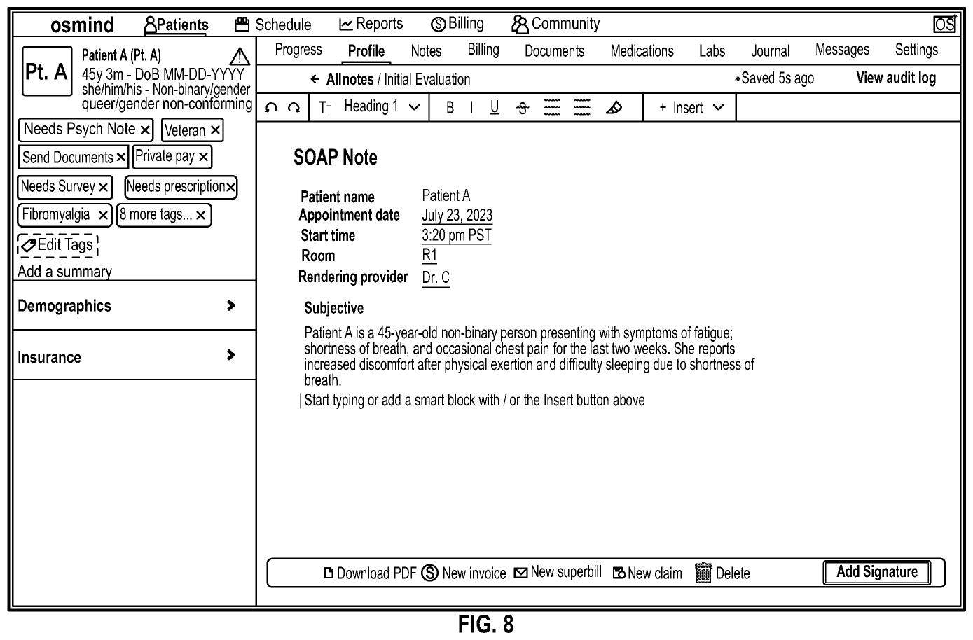

FIG. 8 is an illustration of an embodiment of a provider-facing interface. As shown in FIG. 8, the patient profile may include information regarding the patient. In an embodiment, the patient profile may include a list of treatments that the patient has either completed, is enrolled in, has been enrolled in, or may be prospectively enrolled in. Yet further, each of the treatments on the list of treatments may include a status (e.g., “completed,” “pending,” “withdrawn,” etc.) of the patient's progress with said treatment and/or a treatment adherence probability (e.g., displayed as a percentage of likely completion). Moreover, the provider-facing interface may include at least one tag, wherein said tag may include information such as, how the patient pays for treatment, whether the patient is or is not a veteran, whether the patient requires a prescription, any diseases and/or disorders the patient may be afflicted with, whether the patient requires further evaluation, and/or any future tasks the healthcare provider may need to perform. In an alternative embodiment, the patient profile may include an area to take notes.

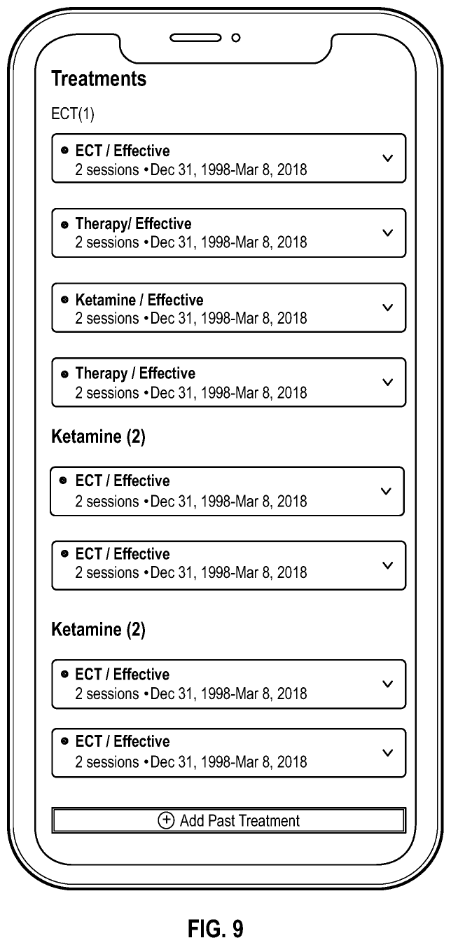

FIG. 9 is an illustration of an embodiment of a provider-facing interface. As shown in FIG. 9, the treatment history interface may display the treatments previously provided or currently provided to the patient. Each of the treatment displays may be interactive, such that a provider may select one or more of the historical treatment instances to reveal information pertaining to said treatment instance. Furthermore, the treatment displays may be organized. In an embodiment, the treatment displays are organized by date and/or time. In a further embodiment, the treatment displays are organized by the type of treatment provided to the patient. The treatment history interface may include an option to add past treatments, wherein adding a past treatment will add said treatment to the treatment displays.

In an embodiment, the desired target variable for a dataset may be an indicator of whether a patient completed the prescribed full initial course (induction) of the particular treatment (e.g., IV ketamine infusions). The full initial course may be defined as completing a predetermined portion of the treatment (e.g., at least 4 infusions within 28 days from the intake evaluation).

In an embodiment, patients who completed the full course may be assigned a 1 and all other patients may be assigned a 0, for the purpose of training statistical models to predict said target.

The algorithm and underlying classifier may be determined by seeking a classifier that predicts the target as accurately as possible. More specifically, methods may be executed to produce a classifier to be as accurate when making predictions for future patients with data that was not used to train the model.

Accordingly, a “test set” may be created by removing a predetermined portion of the sample and holding it out for later testing. The remaining portion of data (the “training set”) may be utilized for a sequence of model training trials. Each trial may test a unique combination of each of the parameters. Parameters may include the type of classifier, the value of regularization strength of the classifier (for example, with a predetermined number of options tested), the probability threshold for predicting a patient as a “completer” (for example, a predetermined number of values tested).

For each of the trials, the classifier may be “fit” to the training data. For example, in “fitting” the classifier attempts to learn the mathematical relationship between the inputs and the target variable. In an embodiment, for each trial, the unique combination of parameters may impact how the model fits to the training data. In such an embodiment, as a result, the fitted models from all the trials may differ from each other, even when all are using the same data for model fitting. The purpose of repeating multiple trials with different parameters may be to discover the combination of parameters that produce the most accurate predictions for “out-of-sample” data (data that the model did not use for fitting).

From the models that were tested, the best model may be selected according to a set of scores collected for each trial. In an embodiment, scores are generated using a process of “cross-validation”. For each trial, the model may fit to a subset of the training data, and the fitted model may be used to predict the remaining portion. On each trial, this process repeats a predetermined number of times (once for each unique portion of training data, with predictions made for the remaining portion), and all predictions may be stored. These predictions may then be compared to the true values to compute a set of scores for each trial.

In an embodiment, the data inputs for the algorithm may be entered into a patient mobile or web application and/or a provider web application. All inputs may be stored in an electronic health record database. In an embodiment, a separate software “container” holds all the necessary programming to convert the raw data stored in the database to the patient's estimated probability of treatment completion. In an embodiment, this conversion follows a sequence of the following steps: each raw data point is converted into a rescaled value; according to the formula provided by the trained algorithm, a multiplier or “weight” is assigned to each input data point to calculate the change in probability associated with that specific input; and all inputs are summed to generate the final probability prediction.

In an embodiment, the algorithm software container stores the probability value in the electronic health record database and then the probability value may be made visible in the patient's chart in the web application of the electronic health record platform.

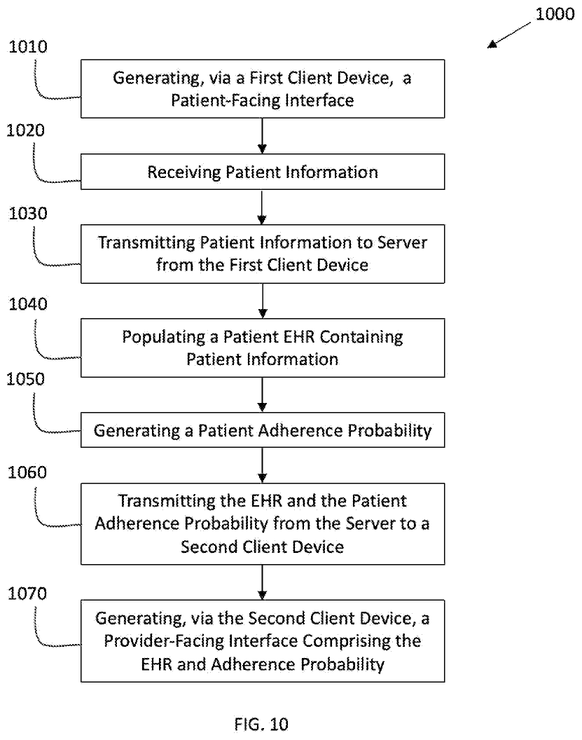

Referring to FIG. 10, the System may include a computer-implemented method 1000. Said method may at step 1010, generate, via a first client device, a patient-facing interface, wherein said interface is configured to receive patient information from a patient. In an embodiment, the method, at step 1020, may receive, via the first client device, the patient information, wherein said information is, at step 1030, transmitted to a server. In another embodiment, the method may, at step 1040, populate, via at least one server processor an EHR of the patient with the patient information. Further, the method, at step 1050, may generate an adherence probability of the patient, via at least one of the at least one server processor and a treatment adherence algorithm, wherein said algorithm may be based on at least a portion of the patient information. Said adherence probability of the patient may be the likelihood the patient will complete a treatment. In a further embodiment, the method may, at step 1060, transmit at least one of the EHR of the patient and the adherence probability of the patient from the server to a second client device. In yet a further embodiment, the method may, at step 1070, generate a provider-facing interface, via the second client device, wherein said interface may be comprised of the EHR of the patient, and wherein the EHR of the patient is comprised of the adherence probability of the patient.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the claims.

What is claimed is:

1. A system for electronic health record (EHR) management and dynamic treatment adherence prediction, the system comprising a server comprising at least one server processor, at least one server database, at least one server memory comprising a set of computer-executable server instructions which, when executed by the at least one server processor, cause the server to:
generate, via a first client device, a patient-facing interface configured to receive patient information from a patient;
receive, via the first client device, the patient information;
transmit, from the first client device to the server, the patient information;
populate, via the at least one server processor, an EHR of the patient with the patient information;
generate, via the at least one server processor, via a treatment adherence algorithm based at least on a portion of the patient information, an adherence probability of the patient,
wherein the adherence probability of the patient is the likelihood that the patient will complete a treatment;
transmit, from the server to a second client device, the EHR of the patient and the adherence probability of the patient;
generate, via the second client device, a provider-facing interface comprising the EHR of the patient, the EHR of the patient comprising the adherence probability of the patient.

2. The system of claim 1, the set of computer-executable server instructions which, when executed by the at least one server processor, further cause the server to:
generate, via the at least one server processor, via a secondary treatment adherence algorithm based at least on a portion of the patient information, a secondary adherence probability of the patient,
wherein the secondary adherence probability of the patient is the likelihood that the patient will complete a secondary treatment;
transmit, from the server to the second client device, the secondary adherence probability of the patient;
generate, via the second client device, the provider-facing interface comprising the EHR of the patient, the EHR of the patient comprising the adherence probability of the patient and the secondary adherence probability of the patient.

3. The system of claim 2, wherein the treatment and the secondary treatment are configured to treat a same ailment.

4. The system of claim 1, wherein the treatment comprises intravenous (IV) ketamine infusions.

5. The system of claim 4, wherein the completeness of the treatment is defined as the patient completing at least four IV ketamine infusions within twenty-eight days from an intake evaluation.

6. The system of claim 1, wherein the treatment adherence algorithm is selected from a group of classifier types comprising Bayesian linear models, hierarchical models, naive Bayes classifiers, and kernel-based methods.

7. The system of claim 1, wherein the patient information is appended by one or more external sources.

8. The system of claim 7, wherein the one or more external sources comprise a digital biomarker-capturing device configured to capture one or more digital biomarkers from the patient.

9. The system of claim 8, wherein the digital biomarker-capturing device is integrated within the first client device.

10. The system of claim 1, the set of computer-executable server instructions which, when executed by the at least one server processor, further cause the server to:
detect, via the server processor, one or more changes to the patient information;
regenerate, triggered by the one or more changes to the patient information, via the at least one server processor, via the treatment adherence algorithm based at least on the portion of the patient information, the adherence probability of the patient.

11. The system of claim 1, the set of computer-executable server instructions which, when executed by the at least one server processor, further cause the server to:
receive, via the second client device, one or more alterations to the EHR of the patient;
transmit, from the second client device to the server, the one or more alterations;
update, via the server processor, the patient information and the EHR based on the one or more alterations;
regenerate, triggered by the one or more alterations to the patient information, via the at least one server processor, via the treatment adherence algorithm based at least on the portion of the patient information, the adherence probability of the patient.

12. The system of claim 1, the set of computer-executable server instructions which, when executed by the at least one server processor, further cause the server to:
generate, via the second client device, a gating pop-up configured to alert a provider to validate the patient information within the EHR.

13. The system of claim 1, the set of computer-executable server instructions which, when executed by the at least one server processor, further cause the server to:
receive a full dataset;
isolate a training dataset and a test dataset from the full dataset;
split the training dataset into one or more training folds and a validation fold;
train, over a plurality of trials, one or more models on each of the one or more training folds for each of one or more parameter configurations correlated to said one or more model, each of the one or more models configured to classify a target variable,
wherein the target variable is an indicator of whether the patient will complete the treatment;
validate, for each of the one or models for a given trial of the plurality of trials, a given model of the one or more models on the validation fold;
compare a training score with a validation score,
wherein the training score is based on the training of the one or more models over the one or more training folds, and
wherein the validation score is based on the validating of the given model on the validation fold;
record classifications scores for each of the one or more models, the classifications scores based on the training score and the validation score for each of the one or more models;
select the treatment adherence algorithm from the one or more models, the treatment adherence algorithm having a top classification score; and
retrain the treatment adherence algorithm on the full dataset.

14. The system of claim 13, wherein at least the training dataset comprises a plurality of variables, wherein each of the one or more models is configured to classify a target variable based on each of the plurality of variables.

15. The system of claim 14, wherein the plurality of variables comprises one or more continuous variables and one or more binary variables, wherein each of the one or more continuous variables is an integer or real number, and wherein each of the one or more binary variables is encoded as 1 if true, −1 if false, and 0 if missing.

16. The system of claim 15, wherein the plurality of variables comprises a normalized population density of a resident zip code, a normalized median income of a resident zip code, a normalized median home price of a resident zip code, a normalized number of total ICD-10 diagnoses, and a normalized number of ICD-10 diagnoses considered as psychiatric conditions.

17. The system of claim 16, wherein the plurality of variables comprises a normalized number of prior patients treated by a clinic with KIT, a normalized proportion of prior patients at the clinic that met a threshold for adherence to KIT, the patient's age at first infusion, the patient's BMI, and a normalized number of days patient had been associated with the clinic prior to their first KIT treatment.

18. The system of claim 17, wherein the plurality of variables comprises a normalized GAD7 composite score and a normalized PHQ9 composite score.

19. The system of claim 18, wherein the plurality of variables comprises a GAD-7 Item 1 Score, a GAD-7 Item 2 Score, a GAD-7 Item 3 Score, a GAD-7 Item 4 Score, a GAD-7 Item 5 Score, a GAD-7 Item 6 Score, a GAD-7 Item 7 Score, a PHQ-9 Item 1 Score, a PHQ-9 Item 2 Score, a PHQ-9 Item 3 Score, a PHQ-9 Item 4 Score, a PHQ-9 Item 5 Score, a PHQ-9 Item 6 Score, a PHQ-9 Item 7 Score, a PHQ-9 Item 8 Score, and a PHQ-9 Item 9 Score.

20. The system of claim 19, wherein the one or more continuous variables comprises sex, relationship status, completion status of intake form, mood disorder diagnosis, anxiety disorder diagnosis, attention disorder diagnosis, pre-visit status, and provider physician status.
